(19) United States
(12) Patent Application Publication
(10) Pub. No.: US 2023/0284966 A1
(43) Pub. Date: Sep. 14, 2023

(54) CAPSULE FOR DIAGNOSIS OF HELICOBACTER PYLORI

(71) Applicants: DAEGU GYEONGBUK INSTITUTE OF SCIENCE AND TECHNOLOGY, Daegu (KR); INDUSTRY-ACADEMIC COOPERATION FOUNDATION, YONSEI UNIVERSITY, Seoul (KR)

(72) Inventors: Suk Ho PARK, Gwangju (KR); Seung Min BANG, Seoul (KR)

(21) Appl. No.: 18/015,996

(22) PCT Filed: Apr. 16, 2021
(86) PCT No.: PCT/KR2021/004790

§ 371 Date: Jan. 13, 2023

(30) Foreign Application Priority Data
Jul. 13, 2020 (KR) 10-2020-0086064

(51) Int. Cl. A61B 5/00; A61B 5/07

(52) U.S. Cl. CPC A61B 5/4216; A61B 5/6861; A61B 5/073; A61B 5/0084; A61B 2560/0219

(57) ABSTRACT

The present invention provides a capsule for diagnosis of Helicobacter pylori including a body section into which a gastrointestinal fluid including the Helicobacter pylori is selectively introduced; a decomposition section that is accommodated in the body section and contains urea; a sensor unit for measuring information on a result of decomposing the urea by Helicobacter pylori in the decomposition section; and a control unit for transmitting the information measured by the sensor unit to the outside.

### TECHNICAL FIELD

The present invention relates to a capsule for diagnosis of Helicobacter pylori, and more particularly, to a capsule for diagnosis of presence or absence of Helicobacter pylori by entering a human stomach.

### BACKGROUND

In general, Helicobacter pylori is a bacterium that infects an inner wall of a person's stomach and is a spiral bacteria having several flagella and is characterized by a slow growth rate and fast movement. The Helicobacter pylori has urease and decomposes urea into ammonia while staying in the gastrointestinal mucosa. As the Helicobacter pylori decomposes the urea into ammonia, gastric acid is partially neutralized to increase a pH value around the Helicobacter pylori existing in the stomach, and thus, the Helicobacter pylori has high viability in the stomach even at a low pH value of gastric acid.

There is a Campylobacter-Like Organism (CLO) test as a method for diagnosing Helicobacter pylori. The CLO test uses urea, phenol red, a buffer, and a bacteriostatic agent to observe that the phenol red changes from yellow to red as urea is decomposed into ammonia by the Helicobacter pylori, and thereby, it is possible to diagnose presence or absence of the Helicobacter pylori.

In this case, according to the known method for diagnosing Helicobacter pylori, there is a problem that causes inconvenience to a patient because a biopsy has to be directly performed through an endoscopic procedure.

### SUMMARY OF INVENTION

### Technical Problem

The present invention is developed to solve the above problems and aims to provide a capsule for diagnosis of Helicobacter pylori which can easily diagnose whether there is Helicobacter pylori in a patient's stomach by simply swallowing the diagnostic capsule by designing a diagnostic device as a capsule type.

### Solution to Problem

The present invention provides a capsule for diagnosis of Helicobacter pylori including a body section into which a gastrointestinal fluid including the Helicobacter pylori is selectively introduced; a decomposition section that is accommodated in the body section and contains urea; a sensor unit for measuring information on a result of decomposing the urea by Helicobacter pylori in the decomposition section; and a control unit for transmitting the information measured by the sensor unit to the outside.

The capsule for diagnosis of Helicobacter pylori according to the present invention may further include a power supply unit that is accommodated in the body section and supplies power to the control unit.

The capsule for diagnosis of Helicobacter pylori according to the present invention may further include a power supply unit that is installed outside the body section and drives the control unit through a radio frequency identification (RFID) technique.

The body section may have an upper portion formed in a partially open shape, the decomposition section may be formed in an inner space of the body section, and the sensor unit and the control unit may be formed in the inner space of the body section and arranged in order below the decomposition section.

The decomposition section may be arranged to be separated from an inlet of the body section.

The decomposition section may further include a buffer and a bacteriostatic agent that inhibits growth and proliferation of Helicobacter pylori.

The sensor unit may measure a pH value of the gastrointestinal fluid that is changed by ammonia generated as the urea is decomposed, and the control unit may transmit the measured pH value to the outside.

The capsule for diagnosis of Helicobacter pylori according to the present invention may further include a magnetic drive unit that is accommodated in the body section and controlled by a magnetic field formed outside.

The capsule for diagnosis of Helicobacter pylori according to the present invention may further include a light source unit that is accommodated in an interior of the body section and illuminates the decomposition section, wherein the decomposition section may further include an acid-base indicator, and the sensor unit may measure image information of the decomposition section illuminated by the light source unit.

The light source unit may be installed on an inner wall of the body section, the decomposition section and the sensor unit may be arranged in an inner space of the body section, and the control unit may transmit the image information formed by the sensor unit to the outside as the light source unit illuminates the decomposition section.

The image information may be one of an actual image of the decomposition section and an RGB value of the decomposition section.

The capsule for diagnosis of Helicobacter pylori according to the present invention may further include a cover section that is installed at an inlet of the body section and removed as the cover section comes into contact with the gastrointestinal fluid.

The cover section may seal the inlet from the outside of the body section.

The cover section may be formed of a pH-sensitive polymer material.

The decomposition section may further include a swelling gel material as the gastrointestinal fluid is absorbed, and the capsule may further include a sealing lid section that is safely placed on the decomposition section and seals an inlet of the body section as the decomposition section expands.

The body section may have an upper portion in which the inlet is formed, the decomposition section may be separated from a lower side of the inlet and is formed in an inner space of the body section, and the sealing lid section may be safely placed on an upper portion of the decomposition section.

The body section may have a plurality of first accommodation spaces formed in the body section and a second accommodation space communicating with the plurality of first accommodation spaces formed in the body section, the decomposition section and the sensor units may respectively include a plurality of decomposition sections and a plurality of sensor units, which are accommodated in the plurality of first accommodation spaces, and the control unit may be accommodated in the second accommodation space, receives information from each of the plurality of sensor units, and transmits the information to the outside.

The body section may have an upper portion in which a plurality of inlets respectively communicating with the plurality of first accommodation spaces are formed and have partition walls that are formed in the body portion and respectively separate the plurality of first accommodation spaces.

Each of the plurality of decomposition sections may further include a gel material that swells as the gel material comes into contact with a gastrointestinal fluid, and the capsule may further include a plurality of sealing lid sections that are respectively accommodated in the plurality of first accommodation spaces, safely placed on the plurality of decomposition sections, and respectively seal the plurality of inlets of the body section as the plurality of decomposition sections each expand.

The capsule for diagnosis of Helicobacter pylori according to the present invention may further include a plurality of cover sections that are respectively installed at the plurality of inlets of the body section and removed as the cover sections come into contact with the gastrointestinal fluid, and a magnetic drive unit that is accommodated in the second accommodation space and controlled by a magnetic field formed outside, wherein the control unit may remove any one of the plurality of cover sections in a state in which the capsule is moved to any one position in a body by the magnetic drive unit, and then remove another one of the plurality of cover sections in a state in which the capsule is moved to another position.

### Advantageous Effects

According to a capsule for diagnosis of Helicobacter pylori of the present invention, by providing a capsule-type device for diagnosis of Helicobacter pylori, it is possible to simply diagnose whether there is Helicobacter pylori in the patient's stomach only by swallowing a capsule for diagnosis, and a distribution of intestinal bacteria can be checked in various positions in the patient's intestine.

### BRIEF DESCRIPTION OF THE DRAWINGS

FIG. 1 is a cross-sectional view of a capsule for diagnosis of helicobacter according to a first embodiment of the present invention.

FIG. 2 is a cross-sectional view of a capsule for diagnosis of helicobacter according to a second embodiment of the present invention.

FIG. 3 is a cross-sectional view of a capsule for diagnosis of helicobacter according to a third embodiment of the present invention.

FIG. 4 is a cross-sectional view of a capsule for diagnosis of helicobacter according to a fourth embodiment of the present invention.

FIG. 5 is a cross-sectional view of a capsule for diagnosis of helicobacter according to a fifth embodiment of the present invention.

FIG. 6 is a cross-sectional view of a capsule for diagnosis of helicobacter according to a sixth embodiment of the present invention.

### BEST MODE FOR INVENTION

Although the present invention is described with reference to the embodiments illustrated in the drawings, which is merely examples, it will be understood by those skilled in the art that various modifications and equivalent other embodiments are possible therefrom. Therefore, the true technical protection scope of the present invention should be determined by the technical idea of the appended claims.

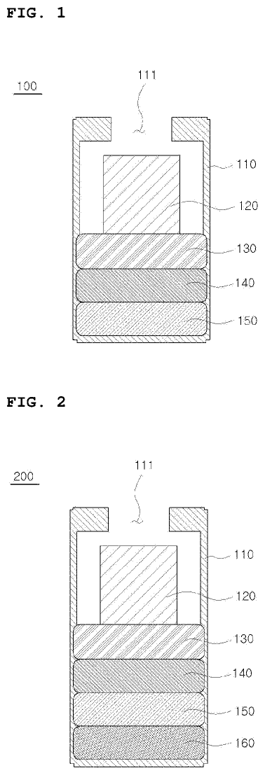

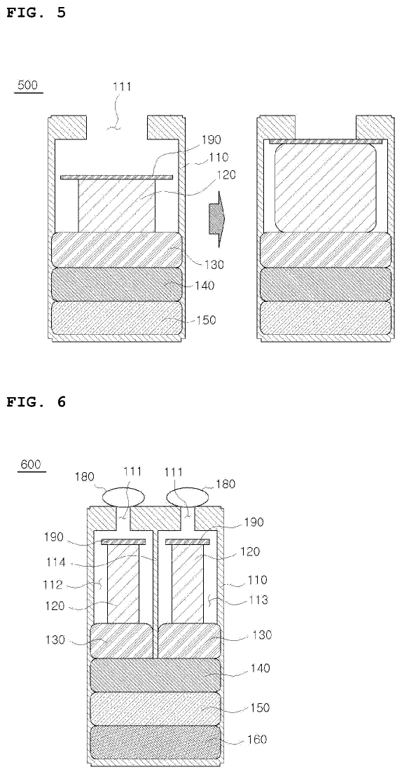

Referring to FIGS. 1 to 6, capsules 100, 200, 300, 400, 500, and 600 for diagnosis of Helicobacter pylori according to the present invention each include a body section 110, a decomposition section 120, a sensor unit 130, a control unit 140, and a power supply unit 150.

The body section 110 has an inlet 111 formed in an upper portion thereof, and Helicobacter pylori (not illustrated) existing in the stomach of a patient is introduced into the interior of the body section 110 through the inlet 111.

The decomposition section 120 is accommodated in the body section 110 and includes urea, a buffer, and a bacteriostatic agent. When Helicobacter pylori is introduced into the body section 110 and comes into contact with the decomposition section 120 to be absorbed therein, urea is decomposed into ammonia. In this case, the buffer allows the Helicobacter pylori absorbed in the decomposition section 120 to survive, and the bacteriostatic agent inhibits growth and proliferation of the Helicobacter pylori.

The sensor unit 130 measures information on a result of decomposition of urea due to Helicobacter pylori in the decomposition section 120. Urea is decomposed into ammonia by Helicobacter pylori, and the sensor unit 130 measures various types of information that can be measured through ammonia which is a product of the decomposition.

The control unit 140 transmits information measured by the sensor unit 130 to the outside, and receives an external control signal to control the decomposition section 120 and the sensor unit 130.

The power supply unit 150 supplies drive power to the control unit 140. The power supply unit 150 can also be installed inside the body section 110 as illustrated in FIGS. 1 to 6, but can also be installed outside the body section 110 (more specifically, outside a patient's body), which is not illustrated in the drawings. When the power supply unit 150 is installed inside the body section 110, the power supply unit 150 is directly connected to the control unit 140 to supply power to the control unit 140. When the power supply unit 150 is installed outside the body section 110, the power supply unit 150 drives the control unit 140 through a radio frequency identification (RFID) technique. The RFID is also called wireless recognition and refers to a recognition system that reads data stored in an object in a non-contact manner by using radio frequency.

The body section 110 has an upper portion formed in an open shape, and the inlet 111 is formed in the upper portion. The decomposition section 120 is formed in an inner space of the body section 110. In addition, the sensor unit 130 and the control unit 140 are arranged in order below the decomposition section 120 and are formed in the inner space of the body section 110. When the power supply unit 150 is installed inside the body section 110 as illustrated in FIGS. 1 to 6, the power supply unit 150 is arranged below the control unit 140 and can be formed in the inner space of the body section 110. In this case, the decomposition section 120 is arranged to be separated from the inlet 111 of the body section 110.

Referring to FIGS. 1, in the capsule 100 for diagnosis of Helicobacter pylori according to the first embodiment of the present invention, the sensor unit 130 measures a pH value of a gastrointestinal fluid changed by ammonia generated as urea is decomposed. In addition, the control unit 140 transmits the measured pH value to the outside.

In the first embodiment of the present invention, an operation sequence of the capsule 100 for diagnosis of Helicobacter pylori will be described as follows.

First, the capsule 100 is delivered to the stomach as a patient swallows the capsule 100. Next, the gastrointestinal fluid enters the capsule 100 to be absorbed by the decomposition section 120. When there is Helicobacter pylori in the gastrointestinal fluid, the Helicobacter pylori decomposes urea in the decomposition section 120 to generate ammonia, whereby a pH value of an inside of the capsule 100 increases, and the sensor unit 130 measures the pH value increased in this way. In addition, the control unit 140 transmits the corresponding pH value to the outside, and a medical staff can determine whether there is Helicobacter pylori in a patient's stomach by looking at the received pH value.

According to the capsule 100 for diagnosis of Helicobacter pylori of the present invention described above, by providing a capsule-type device for diagnosis of Helicobacter pylori, it is possible to simply diagnose whether there is Helicobacter pylori in the patient's stomach only by swallowing a capsule for diagnosis, and in a similar way, a distribution of intestinal bacteria can be checked in various positions in the patient's intestine.

Hereinafter, second to sixth embodiments of the present invention will be described with reference to FIGS. 2 to 6. In this case, parts different from the first embodiment of the present invention will be described.

Referring to FIG. 2, the capsule 200 for diagnosis of Helicobacter pylori according to a second embodiment of the present invention further includes a magnetic drive unit 160. The magnetic drive unit 160 is accommodated in the body section 110 and represents a magnetic body. In the second embodiment of the present invention, a magnetic field can be formed outside a patient's body through a separate magnetic field forming means (not illustrated), and the magnetic drive unit 160 is controlled by an external magnetic field.

In the second embodiment of the present invention, after a patient swallows the capsule 200, a medical staff controls an operation of the magnetic drive unit 160 by adjusting an external magnetic field, adjusts a position of the capsule 200 for diagnosis of Helicobacter pylori in the patient's body through this, and then, analyzes a gastrointestinal fluid flowing into the capsule 200 to diagnose presence or absence of Helicobacter pylori. Therefore, according to the capsule 200 for diagnosis of Helicobacter pylori according to the second embodiment of the present invention, it is possible to measure presence, distribution, and so on of bacteria at a specific desired position in the body.

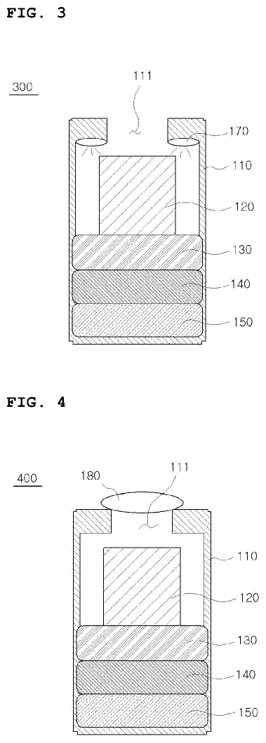

Referring to FIG. 3, the capsule 300 for diagnosis of Helicobacter pylori according to a third embodiment of the present invention further includes a light source unit 170. The light source unit 170 is installed on an upper inner wall of the body section 110 and emits light to the decomposition section 120. The decomposition section 120 further includes an acid-base indicator. Here, the acid-base indicator can be phenol red. The phenol red is yellow in acid and red in neutral. Therefore, when urea is decomposed into ammonia by Helicobacter pylori introduced into the body section 110, the phenol red in the decomposition section 120 also changes from yellow to red.

The sensor unit 130 measures image information of the decomposition section 120 illuminated by the light source unit 170. The control unit 140 transmits image information formed in the sensor unit 130 to the outside as the light source unit 170 illuminates a lower side of the decomposition section 120. Since the light source unit 170 illuminates the lower side, the image information of the decomposition section 120 is formed in the sensor unit 130 arranged below the decomposition section 120, and the image information formed in the sensor unit 130 is transmitted to the control unit 140 arranged below the sensor unit 130.

The image information can be an actual image of the decomposition section 120 illuminated with light. That is, the image information can indicate an actual appearance of the decomposition section 120 as if a picture of the decomposition section 120 is taken by using a camera. Alternatively, the image information can be an RGB value of the decomposition section 120. That is, the sensor unit 130 can measure a color emitted from the decomposition section 120, convert the color into RGB value, and transmit the RGB value to the control unit 140 as the image information.

According to the capsule 300 for diagnosis of Helicobacter pylori according to the third embodiment of the present invention, after urea is decomposed into ammonia by Helicobacter pylori introduced into the body section 110, the sensor unit 130 does not directly measure a pH value inside the capsule 300 but measures image information indicating the pH value of the decomposition section 120 and transmits the image information to the control unit 140, and the control unit 140 transmits the image information of the decomposition section 120 to the outside. In addition, a medical staff diagnoses presence or absence of Helicobacter pylori in a patient's body based on the image information of the decomposition section 120 described above.

Referring to FIG. 4, the capsule 400 for diagnosis of Helicobacter pylori according to a fourth embodiment of the present invention further includes a cover section 180. The cover section 180 is installed at the inlet 111 of the body section 110 and is removed as the cover section 180 comes into contact with a gastrointestinal fluid. More specifically, the cover section 180 can be formed of a pH-sensitive polymer material. In addition, the cover section 180 can be installed to seal the inlet 111 from the outside of the body section 110.

In the fourth embodiment of the present invention, when a patient swallows the capsule 400, the cover section 180 is not removed until the capsule 400 reaches the gastrointestinal fluid, and thus, an undesired material (for example, amylase) can be prevented from being introduced into the capsule 400. In addition, when the capsule 400 swallowed by a patient reaches the stomach, the cover section 180 is removed by the gastrointestinal fluid of high acidity, and then the gastrointestinal fluid can be introduced into the body section 110 through the inlet 111, and thus, a medical staff can more accurately and precisely measure presence or absence of Helicobacter pylori in the gastrointestinal fluid.

The capsule 500 for diagnosis of Helicobacter pylori according to a fifth embodiment of the present invention can further include a sealing lid section 190. The decomposition section 120 can further include a swelling gel material that expands as the gel material absorbs a gastrointestinal fluid. Here, the swelling gel can contain at least one of gelatin, hyaluronic acid, and collagen. The sealing lid section 190 is safely placed on an upper portion of the decomposition section 120 and seals the inlet 111 of the body section 110 as the decomposition section 120 absorbs the gastrointestinal fluid to expand. That is, the sealing lid section 190 can come into contact with an upper inner wall of the body section 110 to seal the inlet 111.

The decomposition section 120 is formed in an inner space of the body section 110, and since an upper surface thereof maintains a state separated from a lower side than the inlet 111, even when the decomposition section 120 absorbs the gastrointestinal fluid to expand, the decomposition section 120 does not interfere with an inner wall of the body section 110.

According to the fifth embodiment of the present invention, when the gastrointestinal fluid flows into the capsule 500 after the capsule 500 reaches a patient's stomach, the decomposition section 120 expands, and the sealing lid section 190 seals the inlet 111 of the body section 110. Accordingly, after the gastrointestinal fluid flows into the capsule 500, an undesirable material (for example, food) can be prevented from flowing into the capsule 500.

Referring to FIG. 6, in the capsule 600 for diagnosis of Helicobacter pylori according to a sixth embodiment of the present invention, the body section 110 includes a plurality of first accommodation spaces 112 and a second accommodation space 113 communicating with the first accommodation space 112. In more detail, a plurality of inlets 111 of the body section 110 are provided separated from each other in left and right directions at an upper portion of the body section 110. The plurality of first accommodation spaces 112 are respectively arranged in the left and right directions and communicate with the plurality of inlets 111 at lower sides of the plurality of inlets 111. In order to divide each of the plurality of first accommodation spaces 112, the body section 110 has a partition wall 114 formed therein. The second accommodation space 113 is arranged below the plurality of first accommodation spaces 112 and communicates with the plurality of first accommodation spaces 112.

FIG. 6 illustrates that there are only two of the first accommodation spaces 112, but this is only an example, and there can be three or more of the first accommodation spaces 112. In correspondence to this, there can be three or more inlets 111, and there can be two or more partition walls 114.

In the sixth embodiment of the present invention, a plurality of decomposition sections 120 and a plurality of sensor units 130 are provided therein and are respectively accommodated in the plurality of first accommodation spaces 112. The control unit 140 is accommodated in the second accommodation space 113. In addition, the control unit 140 receives information from each of the plurality of sensor units 130 and transmits the information to the outside.

The plurality of decomposition sections 120 can each further include a gel material that expands as the gel material comes into contact with a gastrointestinal fluid. In addition, the capsule 600 for diagnosis of Helicobacter pylori according to the sixth embodiment of the present invention can further include a plurality of sealing lid sections 190. The plurality of sealing lid sections 190 are respectively accommodated in the plurality of first accommodation spaces 112 and are respectively safely placed on the plurality of decomposition sections 120. In addition, the plurality of sealing lid sections 190 respectively seal the plurality of inlets 111 as the plurality of decomposition sections 120 respectively expand.

The capsule 600 for diagnosis of Helicobacter pylori according to the sixth embodiment of the present invention can further include a plurality of cover sections 180 and the magnetic drive unit 160. The plurality of cover sections 180 are respectively installed at the plurality of inlets 111 of the body section 110 and are removed as the cover sections 180 come into contact with a gastrointestinal fluid. The magnetic drive unit 160 is accommodated in the second accommodation space 113 and exhibits magnetism and is controlled by a magnetic field formed outside. In a state in which the capsule 600 is moved to any one position inside a body by the magnetic drive unit 160, the control unit 140 removes any one of the plurality of cover sections 180. Thereafter, while the capsule 600 is moved to another position inside the body by the magnetic drive unit 160, the control unit 140 removes another one of the plurality of cover sections 180. In this case, the cover sections 180 are each formed of a wax material, and a heating wire (not illustrated) can be additionally provided to each of the cover sections 180. The control unit 140 can selectively remove the cover sections 180 by controlling the heating wire.

An operation of the sixth embodiment of the present invention will be described in detail as follows. Hereinafter, for the sake of convenience of description, one is designated as A and the other is designated as B.

After a patient swallows the capsule 600 to deliver the capsule 600 to the stomach, the magnetic drive unit 160 moves the capsule 600 to an “A position” due to a change in the external magnetic field. In addition, the control unit 140 removes an “A cover section 180” (for example, the cover section 180 on the left side with reference to FIG. 6) to open an “A first accommodation space 112” (for example, the first accommodation space 112 on the left side with reference to FIG. 6) and allows a gastrointestinal fluid in the “A position” to be introduced into the “A first accommodation space 112”. In this case, an “A decomposition section 120” in the “A first accommodation space 112” is expanded by a gastrointestinal fluid flowing into the “A first accommodation space 112”, and an “A sealing lid section 190” blocks an “A inlet 111”. At the same time, an “A sensor unit 130” measures a pH value or so on of the gastrointestinal fluid flowing into the “A first accommodation space 112”, and the control unit 140 transmits information measured by the “A sensor unit 130” to the outside.

Next, the magnetic drive unit 160 moves the capsule 600 to a “B position” due to a change in the external magnetic field. In addition, the control unit 140 removes a “B cover section 180” (for example, the cover unit 180 on the right side with reference to FIG. 6) to open a “B first accommodation space 112” (for example, the first accommodation space 112 on the right side with reference to FIG. 6) and allows a gastrointestinal fluid in the “B position” to be introduced into the “B first accommodation space 112”. In this case, a “B decomposition section 120” in the “B first accommodation space 112” is expanded by the gastrointestinal fluid flowing into the “B first accommodation space 112”, and a “B sealing lid section 190” blocks a “B inlet 111”. At the same time, a “B sensor unit 130” measures a pH value or so on of the gastrointestinal fluid flowing into the “B first accommodation space 112”, and the control unit 140 transmits information measured by the “B sensor unit 130” to the outside.

In this way, the magnetic drive unit 160 moves the capsule 600 by the number of the first accommodation spaces 112 to another position inside a patient's body to measure bacteria information for each position and transmit the bacteria information to the outside, and a medical staff can check a distribution of intestinal bacteria in various positions in the patient's intestine with the bacteria information.

Meanwhile, the first to sixth embodiments of the present invention described above are not independently implemented, and it will be understood that any one embodiment can be implemented in combination with another embodiment.

1. A capsule for diagnosis of Helicobacter pylori, comprising:
a body section into which a gastrointestinal fluid including the Helicobacter pylori is selectively introduced; a decomposition section that is accommodated in the body section and contains urea;
a sensor unit for measuring information on a result of decomposing the urea by Helicobacter pylori in the decomposition section; and
a control unit for transmitting the information measured by the sensor unit to the outside.

2. The capsule for diagnosis of Helicobacter pylori according to claim 1, further comprising:
a power supply unit that is accommodated in the body section and supplies power to the control unit.

3. The capsule for diagnosis of Helicobacter pylori according to claim 1, further comprising:
a power supply unit that is installed outside the body section and drives the control unit through a radio frequency identification (RFID) technique.

4. The capsule for diagnosis of Helicobacter pylori according to claim 1, wherein
the body section has an upper portion formed in a partially open shape,
the decomposition section is formed in an inner space of the body section, and
the sensor unit and the control unit are formed in the inner space of the body section and arranged in order below the decomposition section.

5. The capsule for diagnosis of Helicobacter pylori according to claim 1, wherein
the decomposition section is arranged to be separated from an inlet of the body section.

6. The capsule for diagnosis of Helicobacter pylori according to claim 1, wherein
the decomposition section further includes a buffer and a bacteriostatic agent that inhibits growth and proliferation of Helicobacter pylori.

7. The capsule for diagnosis of Helicobacter pylori according to claim 1, wherein
the sensor unit measures a pH value of the gastrointestinal fluid that is changed by ammonia generated as the urea is decomposed, and
the control unit transmits the measured pH value to the outside.

8. The capsule for diagnosis of Helicobacter pylori according to claim 1, further comprising:
a magnetic drive unit that is accommodated in the body section and controlled by a magnetic field formed outside.

9. The capsule for diagnosis of Helicobacter pylori according to claim 1, further comprising:
a light source unit that is accommodated in an interior of the body section and illuminates the decomposition section, wherein the decomposition section further includes an acid-base indicator, and
the sensor unit measures image information of the decomposition section illuminated by the light source unit.

10. The capsule for diagnosis of Helicobacter pylori according to claim 9, wherein
the light source unit is installed on an inner wall of the body section,
the decomposition section and the sensor unit are arranged in an inner space of the body section, and
the control unit transmits the image information formed by the sensor unit to the outside as the light source unit illuminates the decomposition section.

11. The capsule for diagnosis of Helicobacter pylori according to claim 9, wherein
the image information is one of an actual image of the decomposition section and an RGB value of the decomposition section.

12. The capsule for diagnosis of Helicobacter pylori according to claim 1, further comprising:
a cover section that is installed at an inlet of the body section and removed as the cover section comes into contact with the gastrointestinal fluid.

13. The capsule for diagnosis of Helicobacter pylori according to claim 12, wherein
the cover section seals the inlet from the outside of the body section.

14. The capsule for diagnosis of Helicobacter pylori according to claim 12, wherein
the cover section is formed of a pH-sensitive polymer material.

15. The capsule for diagnosis of Helicobacter pylori according to claim 1, wherein
the decomposition section further includes a swelling gel material as the gastrointestinal fluid is absorbed, and
the capsule further includes a sealing lid section that is safely placed on the decomposition section and seals an inlet of the body section as the decomposition section expands.

16. The capsule for diagnosis of Helicobacter pylori according to claim 15, wherein
the body section has an upper portion in which the inlet is formed,
the decomposition section is separated from a lower side of the inlet and is formed in an inner space of the body section, and
the sealing lid section is safely placed on an upper portion of the decomposition section.

17. The capsule for diagnosis of Helicobacter pylori according to claim 1, wherein
the body section has a plurality of first accommodation spaces formed in the body section and a second accommodation space communicating with the plurality of first accommodation spaces formed in the body section,
the decomposition section and the sensor units respectively include a plurality of decomposition sections and a plurality of sensor units, which are accommodated in the plurality of first accommodation spaces, and
the control unit is accommodated in the second accommodation space, receives information from each of the plurality of sensor units, and transmits the information to the outside.

18. The capsule for diagnosis of Helicobacter pylori according to claim 17, wherein
the body section has an upper portion in which a plurality of inlets respectively communicating with the plurality of first accommodation spaces are formed and has partition walls that are formed in the body portion and respectively separate the plurality of first accommodation spaces.

19. The capsule for diagnosis of Helicobacter pylori according to claim 18, wherein
each of the plurality of decomposition sections further includes a gel material that swells as the gel material comes into contact with a gastrointestinal fluid, and
the capsule further includes a plurality of sealing lid sections that are respectively accommodated in the plurality of first accommodation spaces, safely placed on the plurality of decomposition sections, and respectively seal the plurality of inlets of the body section as the plurality of decomposition sections each expand.

20. The capsule for diagnosis of Helicobacter pylori according to claim 18, further comprising:
a plurality of cover sections that are respectively installed at the plurality of inlets of the body section and removed as the cover sections come into contact with the gastrointestinal fluid; and
a magnetic drive unit that is accommodated in the second accommodation space and controlled by a magnetic field formed outside,
wherein the control unit removes any one of the plurality of cover sections in a state in which the capsule is moved to any one position in a body by the magnetic drive unit, and then removes another one of the plurality of cover sections in a state in which the capsule is moved to another position.
